(19) United States
(12) Patent Application Publication
(10) Pub. No.: US 2010/0009987 A1
(43) Pub. Date: Jan. 14, 2010

(54) AMINOTHIAZOLE DERIVATIVES AS INHIBITORS OF MARK

(76) Inventors: Michela Bettati, San Secondo (Parma) (IT); Ian Churcher, Hertfordshire (GB); Peter Alan Hunt, Horsham (GB); Victoria Alexandra Steadman, Stansted Mountfitchet (GB)

Correspondence Address: MERCK AND CO., INC, P O BOX 2000, RAHWAY, NJ, 07065-0907, US

(21) Appl. No.: 12/302,164

(22) PCT Filed: Jun. 1, 2007
(86) PCT No.: PCT/GB07/50311

§ 371 Date: Feb. 2, 2009

(30) Foreign Application Priority Data
Jun. 5, 2006 (GB) 0610909.4

(51) Int. Cl. A61K 31/5377; C07D 413/14; C07D 401/14; A61K 31/497; A61P 25/00

(52) U.S. Cl. 514/235.8; 544/121; 544/357; 514/252.11

(57) ABSTRACT

Compounds of formula (I): inhibit microtubule affinity regulating kinase (MARK) and therefore find use in treatment of neurodegenerative diseases associated with hyperphosphorylation of tau.

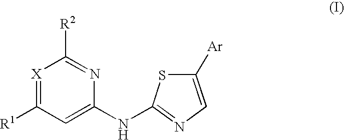

This invention relates to methods and materials for the treatment or prevention of neurodegenerative diseases such as Alzheimer's disease. In particular, there is disclosed a particular class of pyridyl- and pyrimidinylaminothiazole derivatives which selectively inhibit microtubule affinity regulating kinase (MARK).

Alzheimer's disease (AD) is the most common cause of dementia in the elderly and is characterized by a decline in cognitive function, that progresses slowly and results in symptoms such as memory loss and disorientation. Death occurs, on average, 9 years after diagnosis. The incidence of AD increases with age, so that while about 5% of people over the age of 70 are sufferers, this figure increases to 20% of those over 80 years old.

Existing treatments exclusively target the primary symptoms of AD. Diseased neurons may release insufficient or excessive amounts of particular neurotransmitters, and so current drugs are aimed at increasing neurotransmitter levels or at reducing the stimulation of nerve cells by neurotransmitters. Although these drugs provide some improvement in the symptoms of AD, they fail to address the underlying cause of the disease.

The classic clinical and neuropathological features of AD consist of senile or neuritic plaques and tangled bundles of fibers (neurofibrillary tangles) [Verdile, G., et al, Pharm. Res. 50:397-409 (2004)]. In addition, there is a severe loss of neurons in the hippocampus and the cerebral cortex. Neuritic plaques are extracellular lesions, consisting mainly of deposits of β-amyloid peptide (Aβ), surrounded by dystrophic (swollen, damaged and degenerating) neurites and glial cells activated by inflammatory processes. In contrast, neurofibrillary tangles (NFTs) are intracellular clusters composed of a hyperphosphorylated form of the protein tau, which are found extensively in the brain (e.g. mainly in cortex and hippocampus in AD). Tau is a soluble cytoplasmic protein which has a role in microtubule stabilization. Excessive phosphorylation of this protein renders it insoluble and leads to its aggregation into paired helical filaments, which in turn form NFTs.

The amyloid cascade hypothesis proposes that abnormal accumulation of Aβ peptides, particularly Aβ42, initiates a cascade of events leading to the classical symptoms of AD and ultimately, to the death of the patient. There is strong evidence [e.g. Rapoport, M., et al (2002) Proc. Natl. Acad. Sci USA 99:6364-6369] that dysregulation of tau function is a key step in the cascade of Alzheimer's disease pathology leading ultimately to neuronal death. Furthermore, tau mutations and NFTs are found in other dementias in which Aβ pathology is absent, such as frontotemporal dementia, Pick's disease and parkinsonism linked to chromosome 17 (FTDP-17) [Mizutani, T. (1999) Rinsho Shikeigaku 39: 1262-1263]. Also, in AD the frequency of NFTs correlates to the degree of dementia better than that of senile plaques [Arriagada, P. V., et al (1992) Neurology 42:631-639], while significant numbers of amyloid plaques are often found in the brains of non-demented elderly people, suggesting that amyloid pathology on its own is not sufficient to cause dementia. For these reasons, normalization of tau function (in particular prevention of hyperphosphorylation) is seen as a desirable therapeutic goal for the treatment of AD and other dementing conditions.

Tau is a 352-441 amino acid protein encoded by the Mapt (Microtubule-associated protein tau) gene which is widely expressed in the central nervous system (CNS) with localization primarily in axons [Binder et al J. Cell Biol. 1985, 101(4), 1371-1378]. The major function of tau is regulation of the stability of microtubules (MTs), intracellular structural components comprised of tubulin dimers which are integral in regulating many essential cellular processes such as axonal transport and elongation as well as generation of cell polarity and shape. Tau binding to tubulin is a key factor in determining the rates of polymerization/depolymerization (termed dynamic instability) of MTs, and tau is therefore key to the regulation of many essential cellular processes [see, for example, Butner, K. A., Kirschner, M. W. (1991) J.Cell. Biol. 115: 717-730].

Tau is a basic protein with numerous serine and threonine residues, many of which are susceptible to phosphorylation. While normal tau has two to three phosphorylated amino acid residues, hyperphosphorylated tau found in AD and other tauopathies typically has eight or nine phosphorylated residues. A variety of kinases promote phosphorylation of these sites, including proline-directed kinases such as glycogen synthase kinase 3β (GSK3β) and cyclin dependent kinase 5 (cdk5), and non-proline-directed kinases such as protein kinase A (PKA) and calmodulin (CaM) kinase II, which phosphorylate tau at Lys-(Ile/Cys)-Gly-Ser sequences, also known as KXGS motifs. One KXGS motif is found in each of the MT binding repeats. Phosphorylation at these sites is important for the regulation of tau-MT binding and while the degree of phosphorylation is normally low, it has been shown to be increased in brain tissue from AD patients. Phosphorylation of one particular residue within the KXGS motifs, Ser-262 has been shown to be elevated in tau protein extracted from the NFTs in AD [Hasegawa, M. et al (1992) J. Biol. Chem 267:17047-17054] and phosphorylation at this site also appears to dramatically reduce MT binding [Biernat, J. et al. (1993) Neuron 11: 153-163].

Nishimura et al. [Cell 116: 671-682 (2004)] demonstrated that overexpression of the kinase PAR-1 in Drosophila led to enhanced tau-mediated toxicity and an increase in the phosphorylation of tau on Ser-262, Ser-356, and other amino acid residues, including sites phosphorylated by GSK3β and Cdk5. Their findings suggest that PAR-1 kinase acts as a master kinase during the process of tau hyperphosphorylation, with the phosphorylation of the Ser-262 and Ser-356 sites being a prerequisite for the subsequent phosphorylation at downstream sites by other kinases.

The mammalian ortholog of PAR-1 is microtubule affinity-regulating kinase (MARK). There are four MARK isoforms and these form part of the AMP-dependent protein kinase (AMPK) family. Like PAR-1, MARK is thought to phosphorylate tau, perhaps in response to an external insult, such as the disruption of Ca²⁺ homeostasis caused by Aβ, priming it for further phosphorylation events. It is not clear whether the phosphorylation of tau by MARK leads directly to its detachment from MTs or the subsequent phosphorylation events cause detachment. The resulting unbound, hyperphosphorylated tau is delocalized to the somatodendritic compartment and is then cleaved by caspases to form fragments prone to aggregation [Drewes, G. (2004). Trends Biochem. Sci 29:548-555; Gamblin, T. C., et al, (2003) Proc. Natl. Acad. Sci. U.S.A. 100:10032-10037]. These aggregates can grow into filaments, which are potentially toxic, eventually forming the NFTs found in AD.

For these reasons, it is proposed that MARK inhibitors will enable the prevention or amelioration of neurodegeneration in AD and other tauopathies.

In WO 01/17995, US 2005/0228031 and in Bilodeau et al, Biorg. Med. Chem. Lett., 14(2004) 2941-45 and Bilodeau et al, J. Med. Chem., 47 (2004), 6363-72, various aryl- and heteroaryl-aminothiazole derivatives are disclosed as inhibitors of tyrosine kinases (e.g. KDR kinase), implicated in angiogenesis and other cell proliferative processes, but there is no disclosure of the particular compounds of the present invention, or of utility as MARK inhibitors or in the treatment or prevention of tauopathies.

According to the invention, there is provided the use, for the manufacture of a medicament for treatment or prevention of a neurodegenerative disease associated with hyperphosphorylation of tau, of a compound according to formula I:

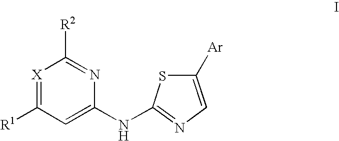

or a pharmaceutically acceptable salt or hydrate thereof; wherein:

X represents CH or N;

R¹ represents NR³R⁴ or OR⁵;

R² represents H or NR³R⁴;

R³ represents H or C₁₋₄alkyl and R⁴ represents CH₂CH₂N(R⁶)₂;

or R³ and R⁴ together complete an N-heterocyclyl group which optionally bears up to 3 substituents selected from halogen, CN, CF₃, CO(O)ₙR⁶ and (CH₂)ₘ(O)ₙR⁶ where m is 1 or 2 and n is 0 or 1;

R⁵ represents C-heterocyclyl which optionally bears up to 2 substituents selected from halogen, CN, CF₃ CO(O)ₙR⁶ and (CH₂)ₘ(O)ₙR⁶ where m is 1 or 2 and n is 0 or 1;

Ar represents phenyl or optionally-benzofused 5- or 6-membered heteroaryl, any of which optionally bears up to 2 substituents independently represented by (CH₂)ₚY where p is 0 or 1 and Y is selected from halogen, CN, CF₃, OR⁶, COR⁶, CO₂R⁶, NR⁶COR⁶, CON(R⁶)₂, N(R⁶)₂, C₁₋₆alkyl, phenyl and pyridyl, said phenyl and pyridyl optionally bearing up to 3 substituents selected from halogen, CN, OH, CF₃, C₁₋₄alkyl and C₁₋₄alkoxy;

R⁶ represents H, C₃₋₆cycloalkyl or C₁₋₆alkyl which optionally bears a substituent selected from halogen, CF₃, CN, OR⁷ or N(R⁷)₂;

or two R⁶ groups attached to the same nitrogen atom complete an N-heterocyclyl group;

R⁷ represents H or C₁₋₄alkyl, or two R⁷ groups attached to the same nitrogen atom complete an N-heterocyclyl group;

where “heterocyclyl” in each case refers to a nonaromatic ring of 5 or 6 members, of which one is N and optionally one other is N, O or S; “N-heterocyclyl” refers to a heterocyclyl group attached via a ring nitrogen atom; and “C-heterocyclyl” refers to a heterocyclyl group attached via a ring carbon atom.

The invention further provides a method for treatment or prevention of a neurodegenerative disease associated with hyperphosphorylation of tau in a human patient, said method comprising administering to that patient an effective amount of a compound of formula I as defined above, or a pharmaceutically acceptable salt or hydrate thereof.

Neurodegenerative diseases associated with hyperphosphorylation of tau include AD, frontotemporal dementia, Pick's disease and parkinsonism linked to chromosome 17 (FTDP-17).

As used herein, the expression “C₁₋ₓalkyl” where x is an integer greater than 1 refers to straight-chained and branched alkyl groups wherein the number of constituent carbon atoms is in the range 1 to x. Particular alkyl groups are methyl, ethyl, n-propyl, isopropyl and t-butyl. Derived expressions such as “C₂₋₆alkenyl”, “hydroxyC₁₋₆alkyl”, “heteroarylC₁₋₆alkyl”, “C₂₋₆alkynyl” and “C₁₋₆alkoxy” are to be construed in an analogous manner. Most suitably, the number of carbon atoms in such groups is not more than 6.

The term “halogen” as used herein includes fluorine, chlorine, bromine and iodine.

The expression “C₃₋₆cycloalkyl” as used herein refers to nonaromatic monocyclic hydrocarbon ring systems comprising from 3 to 6 ring atoms. Examples include cyclopropyl, cyclobutyl, cyclopentyl and cyclohexyl.

For use in medicine, the compounds of formula I may be in the form of pharmaceutically acceptable salts. Other salts may, however, be useful in the preparation of the compounds of formula I or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, benzenesulphonic acid, fumaric acid, maleic acid, succinic acid, acetic acid, benzoic acid, oxalic acid, citric acid, tartaric acid, carbonic acid or phosphoric acid. Alternatively, where the compound of the invention carries an acidic moiety, a pharmaceutically acceptable salt may be formed by neutralization of said acidic moiety with a suitable base. Examples of pharmaceutically acceptable salts thus formed include alkali metal salts such as sodium or potassium salts; ammonium salts; alkaline earth metal salts such as calcium or magnesium salts; and salts formed with suitable organic bases, such as amine salts (including pyridinium salts) and quaternary ammonium salts.

When the compounds useful in the invention have one or more asymmetric centres, they may accordingly exist as enantiomers. Where the compounds according to the invention possess two or more asymmetric centres, they may additionally exist as diastereoisomers. It is to be understood that all such isomers and mixtures thereof in any proportion are encompassed within the scope of the present invention.

When a compound useful in the invention is capable of existing in tautomeric keto and enol forms, both of said forms are considered to be within the scope of the invention.

A nitrogen atom forming part of a heteroaryl ring may be in the form of the N-oxide. A sulphur atom forming part of a nonaromatic heterocycle may be in the form of the S-oxide or S,S-dioxide.

A heteroaryl group may be attached to the remainder of the molecule via a ring carbon or a ring nitrogen, provided that this is consistent with preservation of aromaticity.

In formula I, X represents CH or N. In a particular embodiment, H is CH. In another particular embodiment, X is N and R² is NR³R⁴.

R¹ represents NR³R⁴ or OR⁵. In a particular embodiment, R¹ represents NR³R⁴. In a further embodiment, R¹ and R² both independently represent NR³R⁴.

In one embodiment, R³ represents H or C₁₋₄alkyl and R⁴ represents CH₂CH₂N(R⁶)₂, where R⁶ is as defined previously. In a specific example of this embodiment NR³R⁴ represents NMeCH₂CH₂NMe₂. In an alternative embodiment, NR³R⁴ represents an optionally-substituted N-heterocyclyl group as defined previously, e.g. an optionally-substituted piperidine, piperazine, morpholine or thiomorpholine group. Examples include 4-fluoropiperidin-1-yl, morpholin-4-yl, 2-(hydroxymethyl)morpholin-4-yl, 2,6-dimethylmorpholin-4-yl, 1,1-dioxothiomorpholin-4-yl and piperazin-1-yl which is optionally substituted in the 4-position with CF₃, CO(O)ₙR⁶ or (CH₂)ₘ(O)ₙR⁶ where m is 1 or 2 and n is 0 or 1. Examples of suitable 4-substituents include methyl, 2,2,2-trifluoroethyl, t-butoxycarbonyl, 2-(2-hydroxyethoxy)ethyl and 3-(morpholin-4-yl)propyl.

When R¹ represents OR⁵, R⁵ represents optionally-substituted C-heterocyclyl as defined previously, for example piperidin-4-yl which is optionally substituted in the 1-position with CF₃, CO(O)ₙR⁶ or (CH₂)ₘ(O)ₙR⁶ where m is 1 or 2 and n is 0 or 1.

Ar represents phenyl or 5- or 6-membered heteroaryl, any of which are optionally substituted as defined previously. Heteroaryl groups represented by Ar may be benzo-fused, in which case attachment may be via the fused benzene ring or via the heteroaryl ring itself. Examples of suitable heteroaryl rings include pyridine, thiazole, thiophene, quinoline and benzoxadiazole. In a particular embodiment, Ar represents phenyl or pyridyl (in particular 4-pyridyl) which is optionally-substituted as defined previously. Preferably, the phenyl or heteroaryl represented by Ar bears at most one substituent represented by (CH₂)ₚY where p is 0 or 1 and Y is selected from halogen, CN, CF₃, OR⁶, COR⁶, CO₂R⁶, NR⁶COR⁶, CON(R⁶)₂, N(R⁶)₂, C₁₋₆alkyl, phenyl and pyridyl, said phenyl and pyridyl optionally bearing up to 3 substituents selected from halogen, CN, OH, CF₃, C₁₋₄alkyl and C₁₋₄alkoxy. In one embodiment, when p is 1, Y represents CN or OH. In a further embodiment, p is 0 and Y represents C₁₋₆alkyl (such as methyl), COR⁶, CON(R⁶)₂ or N(R⁶)₂ where R⁶ is as defined previously. R⁶ is very suitably H, C₁₋₄alkyl (such as methyl, ethyl or propyl), C₃₋₆cycloalkyl (such as cyclopentyl), 2,2,2-trifluoroethyl, 2-aminoethyl, 2-hydroxyethyl, or 2-(dimethylamino)ethyl.

A subset of the compounds suitable for use in the invention consists of the compounds of formula II

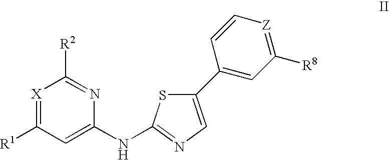

and pharmaceutically acceptable salts or hydrates thereof; wherein;

Z represents N or CR⁹;

R⁸ represents H or (CH₂)ₚY;

R⁹ represents H or (CH₂)ₚY with the proviso that R⁸ and R⁹ do not both represent (CH₂)ₚY;

and p, X, Y R¹ and R² have the same definitions particular identities as described previously.

Specific examples of compounds in accordance with formula II include those in which X is CH, R² is H, R¹ is 4-methylpiperazin-1-yl and Z, R⁸ and (where relevant) R⁹ are as indicated in Table 1:

TABLE 1

| Z | R⁸ | R⁹ |
|---|---|---|
| CR⁹ | NHAc | H |
| CR⁹ | H | CN |
| N | Me | — |
| CR⁹ | H | OH |
| CR⁹ | H | Ac |
| CR⁹ | H | CH₂OH |
| CR⁹ | H | F |
| CR⁹ | NH₂ | H |
| CR⁹ | CH₂OH | H |
| CR⁹ | H | CH₂CN |
| CR⁹ | CONHCH₂CH₂NH₂ | H |
| CR⁹ | CONHCH₂CF₃ | H |
| CR⁹ | CONHCH₂CH₂NMe₂ | H |
| CR⁹ | CONHcyclopentyl | H |
| CR⁹ | CONH₂ | H |
| CR⁹ | H | CONHCH₂CF₃ |
| CR⁹ | CONHCH₂CH₂CH₃ | H |
| CR⁹ | H | Br |
| CR⁹ | 3-pyridyl | H |
| CR⁹ | CONHCH₂CH₂OH | H |
| CR⁹ | CONMeCH₂CH₂NMe₂ | H |
| CR⁹ | CONMe₂ | H |
| CR⁹ | H | CONHCH₂CH₂OH |
| N | NHCH₂CH₂NH₂ | — |
| N | CONHCH₂CH₂NH₂ | — |
| N | morpholin-4-yl | — |
| N | Ph | — |
| N | H | — |

A subset of the compounds of formula II consists of the compounds of formula III:

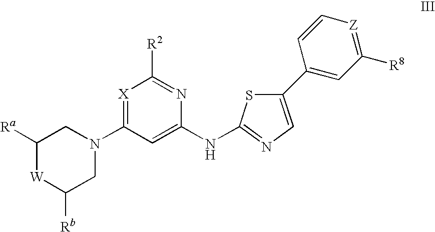

and pharmaceutically acceptable salts and hydrates thereof; wherein:

W represents O, S, SO₂, NR^{c}, CH₂, CHF or CHCF₃;

R^{a} and R^{b} independently represent H, halogen, CN, CF₃, or (CH₂)ₘ(O)ₙR⁶;

R^{c} represents H, CF₃, CO(O)ₙR⁶ or (CH₂)ₘ(O)ₙR⁶;

and X, Z, Ar, m, n, R², R⁶ and R⁸ have the same definitions and particular identities as described previously.

In formula III, R^{a} and R^{b} typically independently represent H, C₁₋₄alkyl (such as methyl) or hydroxyC₁₋₄alkyl (such as hydroxymethyl), and preferably are both H when W represents NR^{c}. Suitable identities for R^{c} include methyl, 2,2,2-trifluoroethyl, t-butoxycarbonyl, 2-(2-hydroxyethoxy)ethyl and 3-(morpholin-4-yl)propyl.

Specific examples of compounds in accordance with formula III include those in which Z represents N and R⁸ represents H (or methyl where so indicated) and W, R^{a}, R^{b}, X and R² are as indicated in table 2:

TABLE 2

| W | R^{a} | R^{b} | X | R² |
|---|---|---|---|---|
| N—(CH₂)₃-(morpholin-4-yl) | H | H | CH | H |
| O | Me | Me | CH | H |
| NH | H | H | CH | H |
| NCH₂CH₂OCH₂CH₂OH | H | H | CH | H |
| N-Boc | H | H | CH | H |
| O | H | H | CH | H |
| SO₂ | H | H | CH | H |
| O | CH₂OH | H | CH | H |
| CHF | H | H | CH | H |
| NCH₂CF₃ | H | H | CH | H |
| NMe | H | H | CH | piperazin-1-yl |
| *NMe | H | H | CH | piperazin-1-yl |
| NH | H | H | CH | 4-Me-piperazin-1-yl |
| N—(CH₂)₃-(morpholin-4-yl) | H | H | CH | 4-Me-piperazin-1-yl |
| NMe | H | H | CH | 4-Me-piperazin-1-yl |
| NCH₂CF₃ | H | H | CH | 4-Me-piperazin-1-yl |
| *NMe | H | H | CH | 4-Boc-piperazin-1-yl |
| O | Me | Me | CH | 4-Me-piperazin-1-yl |
| NMe | H | H | N | 4-Me-piperazin-1-yl |
| O | H | H | N | 4-Me-piperazin-1-yl |

*R⁸ = Me
Boc = t-butoxycarbonyl.

Compounds of formulae II and III and the pharmaceutically acceptable salts or hydrates thereof are believed to be novel, and constitute a further aspect of the invention.

The invention further extends to a pharmaceutical composition comprising a compound of formula II or formula III or a pharmaceutically acceptable salt or hydrate thereof in a pharmaceutically acceptable carrier.

The invention further provides a compound of formula II or formula III or a pharmaceutically acceptable salt or hydrate thereof for use in medicine.

Compounds of formula I may be prepared by methods disclosed in WO 01/17995 or simple adaptations thereof. In a particular route to compounds of formula I, a boronic acid derivative Ar—B(OH)₂ or an ester derivative thereof such as the pinacolate is coupled with a compound of formula (1a):

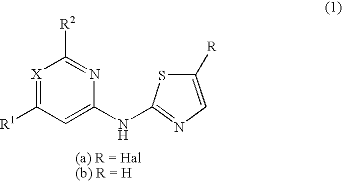

where Hal represents Cl, Br or I and R¹, R² X and Ar have the same meanings as before. The reaction takes place under standard conditions of Suzuki coupling, e.g. in an ether solvent such as dimethoxyethane or dioxan with microwave heating in the presence of a base such as potassium carbonate or potassium phosphate and a Pd(0) catalyst such as Pd(PPh₃)₄. Compounds (1a) are obtainable by halogenation of compounds (1b), e.g. using bromine in acetic acid at ambient temperature.

Compounds (1b) are obtainable by reaction of 2-chlorothiazole with an amine of formula (2):

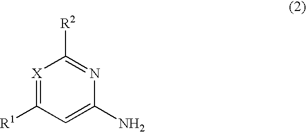

where X, R¹ and R² have the same meanings as before. The reaction takes place in the presence of sodium hydride in an aprotic solvent such as dioxin, e.g. with heating to about 90° C.

Amines of formula (2) in which X is CH may be obtained by treatment of chloropyridine derivatives (3a) with R¹—H:

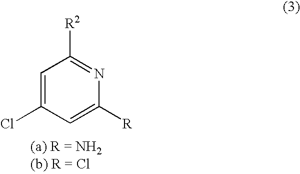

where R¹ and R² have the same meanings as before. The reaction takes place in an inert solvent such as N-methylpyrrolidone at elevated temperature (e.g. about 225° C. under microwave heating), preferably in the presence of base when R¹ represents R⁵O. Amines (3a) are available by treatment of chlorides (3b) with benzophenone imine, sodium t-butoxide, Pd₂dba₃ and BINAP in refluxing toluene, then with hydroxylamine. Chlorides (3b) in which R² is NR³R⁴ are obtained from reaction of 2,4,6-trichloropyridine with R³R⁴NH under the same conditions as reaction of (3a) with R¹—H.

Amines of formula (2) in which X is N and R² is H may be prepared by sequential treatment of 4,6-dichloropyrimidine with ammonia and R¹—H. Amines of formula (2) in which X is N and R² is NR³R⁴ may be prepared by similar treatment of 4,6-dichloro-2-(methylthio)pyrimidine with ammonia and R¹—H, followed by oxidation of the thioether to the corresponding sulfone and displacement of the methylsulfonyl group using R³R⁴NH.

An alternative route to the compounds of formula I involves reaction of an amine (2) with a chlorothiazole derivative (4a):

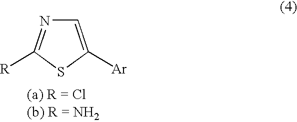

where Ar has the same meaning as before, under similar conditions to the above-described reaction between amines (2) and 2-chlorothiazole itself. Chlorides (4a) are available by reaction of amines (4b) with CuCl₂ and t-BuNO₂ in acetonitrile at ambient temperature.

Amines (4b) may be prepared by reaction of arylacetaldehydes ArCH₂CHO with bromine (e.g. at −10° C. in dichloromethane), followed by reaction of the resulting benzyl bromide with thiourea (e.g. in refluxing ethanol).

Alternatively, amines (4b) can be obtained by coupling of Ar-Hal (where Hal represents Cl, Br or I) with 2-aminothiazole (protected as the N-pivaloyl derivative). The coupling takes place at elevated temperature (e.g. about 125° C.) in a polar solvent such as dimethylacetamide in the presence of base (such as potassium acetate) and a Pd(0) catalyst (such as Pd(PPh₃)₄). This route is particularly suitable when Ar represents 4-pyridyl.

It will be readily apparent that individual compounds in accordance with formula I may be converted into other compounds in accordance with formula I by means of standard techniques of synthetic chemistry familiar to those skilled in the art. For example, compounds in which Ar bears a halogen substituent may be reacted with the appropriate boronic acid derivative under Suzuki conditions to provide the corresponding phenyl- or pyridyl-substituted derivative. Similarly, compounds in which Ar bears a halogen substituent may be reacted with (R⁶)₂NH and CO gas to provide the corresponding compound bearing a CON(R⁶)₂ substituent, the reaction taking place in the presence o base and a Pd(II) catalyst in dimethylacetamide in a sealed tube.

Where they are not themselves commercially available, the starting materials and reagents described above may be obtained from commercially available precursors by means of well known synthetic procedures and/or the methods disclosed in the Examples section herein.

Where the above-described processes for the preparation of the compounds of use in the invention give rise to mixtures of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques such as preparative HPLC, or the formation of diastereomeric pairs by salt formation with an optically active acid, such as di-p-toluoyl-D-tartaric acid and/or di-p-toluoyl-L-tartaric acid, followed by fractional crystallization and regeneration of the free base. The compounds may also be resolved by formation of diastereomeric esters or amides, followed by chromatographic separation and removal of the chiral auxiliary.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art. For example, the secondary amine group in compounds (1) is preferably converted to the N-acetyl derivative prior to reaction with ArB(OH)₂.

The compounds of formula I are suitably administered to patients in the form a pharmaceutical composition comprising the active ingredient (i.e. the compound of formula I or pharmaceutically acceptable salt or hydrate thereof) and a pharmaceutically acceptable carrier.

Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. Tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

In one embodiment of the invention, the compound of formula I is administered to a patient suffering from AD, FTDP-17, Pick's disease or frontotemporal dementia, preferably AD.

In an alternative embodiment of the invention, the compound of formula I is administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organization, 1992, 64-5). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al., Arch. Neurol., 56 (1999), 303-8. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Within this embodiment, the compound of formula I is advantageously administered to patients who suffer impaired memory function but do not exhibit symptoms of dementia. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus.

In a particular embodiment of the invention, the compound of formula I is administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42).

A genetic predisposition (especially towards early onset AD) can arise from point mutations in one or more of a number of genes, including the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment in accordance with the invention, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assoc'n. 18 (1993), 2386-2391). The MMSE is a brief quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J. Psychiatry, 141 (1984), 1356-64).

For treating or preventing Alzheimer's disease, a suitable dosage level is about 0.01 to 250 mg/kg per day, preferably about 0.01 to 100 mg/kg per day, and more preferably about 0.05 to 50 mg/kg of body weight per day, of the active compound. The compounds may be administered on a regimen of 1 to 4 times per day. In some cases, however, a dosage outside these limits may be used.

The compound of formula I optionally may be administered in combination with one or more additional compounds known to be useful in the treatment or prevention of AD or the symptoms thereof. Such additional compounds thus include cognition-enhancing drugs such as acetylcholinesterase inhibitors (e.g. donepezil and galanthamine), NMDA antagonists (e.g. memantine) or PDE4 inhibitors (e.g. Ariflo™ and the classes of compounds disclosed in WO 03/018579, WO 01/46151, WO 02/074726 and WO 02/098878). Such additional compounds also include cholesterol-lowering drugs such as the statins, e.g. simvastatin. Such additional compounds similarly include compounds known to modify the production or processing of Aβ in the brain (“amyloid modifiers”), such as compounds which modulate the secretion of Aβ (including γ-secretase inhibitors, γ-secretase modulators and β-secretase inhibitors), compounds which inhibit the aggregation of Aβ, and antibodies which selectively bind to Aβ. Such additional compounds further include growth hormone secretagogues, e.g. as described in WO 2004/080459.

In this embodiment of the invention, the amyloid modifier may be a compound which inhibits the secretion of Aβ, for example an inhibitor of γ-secretase (such as those disclosed in WO 01/53255, WO 01/66564, WO 01/70677, WO 01/90084, WO 01/77144, WO 02/30912, WO 02/36555, WO 02/081435, WO 02/081433, WO 03/018543, WO 03/013506, WO 03/013527, WO 03/014075, WO 03/093251, WO 03/093252, WO 03/093253, WO 03/093264, WO 2004/031137, WO 2004/031138, WO 2004/031139, WO 2004/039370, WO 2004/039800, WO 2004/101538, WO 2004/101539 and WO 2005/030731), or a β-secretase inhibitor (such as those disclosed in WO 03/037325, WO 03/030886, WO 03/006013, WO 03/006021, WO 03/006423, WO 03/006453, WO 02/002122, WO 01/70672, WO 02/02505, WO 02/02506, WO 02/02512, WO 02/02520, WO 02/098849 and WO 02/100820), or any other compound which inhibits the formation or release of Aβ including those disclosed in WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621.

Within this embodiment, the amyloid modifier is advantageously a γ-secretase inhibitor, preferred examples of which include a compound of formula XI:

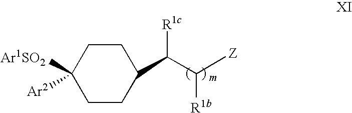

wherein the variables are as defined in WO 03/018543. Preferred examples include those defined by formula XIa:

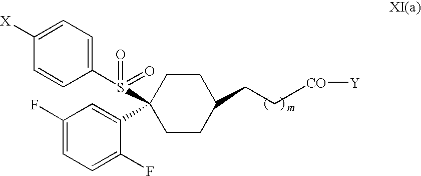

and the pharmaceutically acceptable salts thereof, wherein m is 0 or 1, X is Cl or CF₃, and Y is OH, OC₁₋₆alkyl, NH₂ or NHC₁₋₆alkyl. Particular examples include those in which m is 1 and Y is OH (or the sodium salts thereof), and those in which m is 0 and Y is NH₂ or NHC₁₋₆alkyl.

Another preferred class of γ-secretase inhibitors for use in this embodiment of the invention is that defined by formula XII:

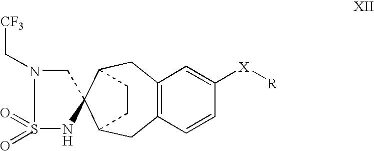

and the pharmaceutically acceptable salt thereof;
wherein the variables are as defined in WO 03/093252.

X is very aptly 5-substituted-thiazol-2-yl, 5-substituted-4-methylthiazol-2-yl, 5-substituted-1-methylpyrazol-3-yl, 1-substituted-imidazol-4-yl or 1-substituted-1,2,4-triazol-3-yl. Preferably, R represents optionally-substituted phenyl or heteroaryl such as phenyl, monohalophenyl, dihalophenyl, trihalophenyl, cyanophenyl, methylphenyl, methoxyphenyl, trifluoromethylphenyl, trifluoromethoxyphenyl, pyridyl, monohalopyridyl and trifluoromethylpyridyl, wherein “halo” refers to fluoro or chloro. Particularly preferred identities of R—X— include 5-(4-fluorophenyl)-1-methylpyrazol-3-yl, 5-(4-chlorophenyl)-1-methylpyrazol-3-yl and 1-(4-fluorophenyl)imidazol-4-yl. Such compounds may be prepared by methods disclosed in WO 03/093252.

Alternatively, the amyloid modifier may be a compound which modulates the action of γ-secretase so as to selectively attenuate the production of Aβ (1-42). Compounds reported to show this effect include certain non-steroidal antiinflammatory drugs (NSAIDs) and their analogues (see WO 01/78721 and US 2002/0128319 and Weggen et al Nature, 414 (2001) 212-16; Morihara et al, J. Neurochem., 83 (2002), 1009-12; and Takahashi et al, J. Biol. Chem., 278 (2003), 18644-70), and compounds which modulate the activity of PPARγ and/or PPARδ (WO 02/100836). Further examples of γ-secretase modulators are disclosed in WO 2005/054193, WO 2005/013985 and WO 2005/108362.

Alternatively, the amyloid modifier may be a compound which inhibits the aggregation of Aβ. Suitable examples include chelating agents such as clioquinol (Gouras and Beal, Neuron, 30 (2001), 641-2) and the compounds disclosed in WO 99/16741, in particular that known as DP-109 (Kalendarev et al, J. Pharm. Biomed. Anal., 24 (2001), 967-75). Other inhibitors of Aβ aggregation suitable for use in the invention include the compounds disclosed in WO 96/28471, WO 98/08868 and WO 00/052048, including the compound known as Apan™ (Praecis); WO 00/064420, WO 03/017994, WO 99/59571 and the compound known as Alzhemed™ (Neurochem); WO 00/149281 and the compositions known as PTI-777 and PTI-00703 (ProteoTech); WO 96/39834, WO 01/83425, WO 01/55093, WO 00/76988, WO 00/76987, WO 00/76969, WO 00/76489, WO 97/26919, WO 97/16194, and WO 97/16191.

Alternatively, the amyloid modifier may be an antibody which binds selectively to Aβ. Said antibody may be polyclonal or monoclonal, but is preferably monoclonal, and is preferably human or humanized. Preferably, the antibody is capable of sequestering soluble Aβ from biological fluids, as described in WO 03/016466, WO 03/016467, WO 03/015691 and WO 01/62801. Suitable antibodies include humanized antibody 266 (described in WO 01/62801) and the modified version thereof described in WO 03/016466. Suitable antibodies also include those specific to Aβ-derived diffusible ligands (ADDLS), as disclosed in WO 2004/031400.

As used herein, the expression “in combination with” requires that therapeutically effective amounts of both the compound of formula I and the additional compound are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. When the additional compound is an antibody, it will typically be administered parenterally and separately from the compound of formula I.

In a further aspect, the invention provides the combination of a compound of formula I and a compound of formula XI(a) or a pharmaceutically acceptable salt thereof for use in treatment or prevention of Alzheimer's disease. Said use may involve the simultaneous or separate administration of the respective compounds to a patient in need of such treatment or prevention.

In a further aspect, the invention provides a pharmaceutical composition comprising, in a pharmaceutically acceptable carrier, a compound of formula I and a compound of formula XI(a) or a pharmaceutically acceptable salt thereof. Preferably, the pharmaceutical composition is in a unit dose form suitable for oral administration, such as a tablet or a capsule.

### EXAMPLES

### MARK 3 Assay

MARK3 activity was assayed in vitro using a Cdc25C biotinylated peptide substrate (Cell Signaling Technologies). The phosphopeptide product was quantitated using a Homogenous Time-Resolved Fluorescence (HTRF) assay system (Park et al., 1999, Anal. Biochem. 269:94-104). The reaction mixture contained 50 mM HEPES/Tris-HCl, pH 7.4; 10 mM NaCl, 5 mM MgCl₂, 0.2 mM NaVO₄, 5 mM β-glycerol phosphate, 0.1% Tween-20, 2 mM dithiothreitol, 0.1% BSA, 10 μM ATP, 1 μM peptide substrate, and 10 nM recombinant MARK3 enzyme (University of Dundee) in a final volume of 12 μl. The buffer additionally contained protease inhibitor cocktail (Roche EDTA-free, 1 tab per 50 ml). The kinase reaction was incubated for 2 hours at 25° C., and then terminated with 3 μl Stop/Detection Buffer (50 mM HEPES, pH 7.0, 16.6 mM EDTA, 0.5M KF, 0.1% Tween-20, 0.1% BSA, 2 μg/ml SLX^{ent} 665 (CISBIO), and 2 μg/ml Eu³ cryptate label antibody (CISBIO)). The reaction was allowed to equilibrate overnight at 0° C., and relative fluorescent units were read on an HTRF enabled plate reader (e.g. TECAN GENios Pro). Inhibitor compounds were assayed in the reaction described above to determine compound IC50s. Aliquots of compound dissolved in DMSO were added to the reaction wells in a third-log dilution series covering a range of 1 nM to 10 μM. Relative phospho substrate formation, read as HTRF fluorescence units, was measured over the range of compound concentrations and a titration curve generated.

The compounds listed below gave IC50 values of 1 μM or less, typically 500 nM or less, and in preferred cases 50 nM less, in the above assay.

Schemes 1-4 are representative of the methods used to prepare the compounds suitable for use in the invention.

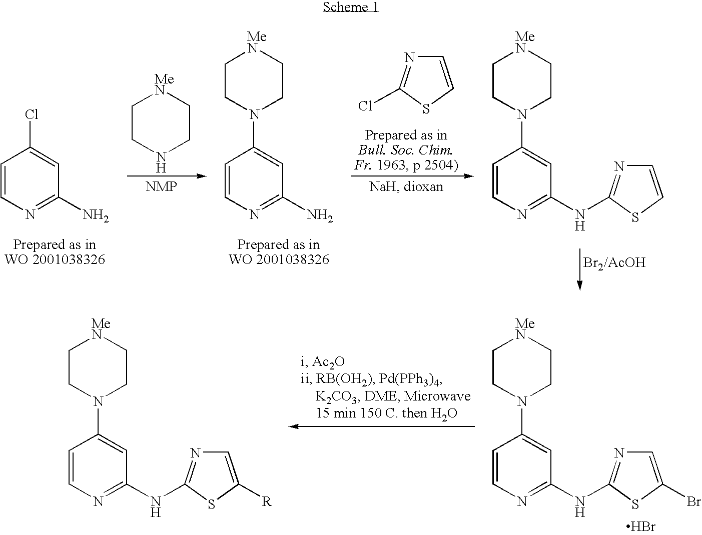

### Scheme 1—Step 1:

2-Amino-4-chloropyridine (1.65 g, prepared as in WO 2001/038326) and N-methylpiperazine (4.2 ml) were dissolved in NMP (15 ml) and sealed in a 20 ml microwave vial. The reaction was heated to 225° C. for 1500 seconds in a microwave apparatus then cooled. Solvent was removed in vacuo then residue taken up in water (40 ml). 4N NaOH (15 ml) was added and the resultant precipitate collected, washed with ether and dried in vacuo to yield the desired 4-(4-methylpiperazin-1-yl)pyridin-2-amine.

Scheme 1—Step 2:

The 4-(4-methylpiperazin-1-yl)pyridin-2-amine from the foregoing step (11.6 g, 60 mmol) was suspended in dry dioxan (250 ml) and sodium hydride (9.7 g of 60% dispersion in mineral oil, 4 eq.) added portionwise over 20 minutes. Stirred further 10 minutes at end of addition then 2-chlorothiazole (9.4 g, 1.3 eq., prepared as in Bull. Soc. Chim. Fr. 1963, p 2504) was added and the reaction heated to 90° C. for 3.5 hours. The mixture was cooled and the solvent removed in vacuo. Water (60 ml) was then slowly, cautiously added with cooling (CAUTION: exotherm and effervescence). At the end of the addition, the mixture was diluted with further water (200 ml) and EtOAc (300 ml) and the resultant solid filtered and washed with ether. Further trituration of the solid with ether afforded the desired 4-(4-methylpiperazin-1-yl)-N-1,3-thiazol-2-ylpyridin-2-amine (11.9 g).

Scheme 1—Step 3:

To a solution of 4-(4-methylpiperazin-1-yl)-N-1,3-thiazol-2-ylpyridin-2-amine from the foregoing step (11.84 g, 43 mmol) in glacial acetic acid (200 ml) at room temperature was added dropwise over 10 minutes bromine (2.4 ml, 1.1 eq.). The reaction was stirred for 1 hour at the end of the addition then ether (300 ml) was added and stirring continued for a further 10 minutes. The mixture was filtered and the solid washed well with ether to afford the desired N-(5-bromo-1,3-thiazol-2-yl)-4-(4-methylpiperazin-1-yl)pyridin-2-amine hydrobromide (13.93 g). The free base was available by partitioning between DCM and 1N NaOH, followed by drying and evaporating the organic layer.

Scheme 1—Step 4:

Acetic anhydride (3 ml) was added to N-(5-bromo-1,3-thiazol-2-yl)-4-(4-methylpiperazin-1-yl)pyridin-2-amine from the foregoing step (0.25 g, 0.7 mmol) and heated to 100° C. for 2 h until a clear solution was obtained. The solvent was evaporated under reduced pressure and the residue azeotroped with toluene. The residue was then diluted with ethyl acetate (20 ml), washed with a saturated solution of NaHCO₃ (30 ml) and brine (30 ml), dried over MgSO₄ and concentrated to yield the desired product (0.28 g, quantitative): δ_{H} (500 MHz, CDCl₃): 8.29 (1H, d, J=6.1 Hz), 7.32 (1H, s), 6.77 (1H, dd, J=6.01 and 2.4 Hz), 6.71 (1H, d, J=2.4 Hz), 3.42-3.40 (4H, m), 2.54-2.52 (4H, m), 2.35 (3H, s), 2.10 (3H, s).

Scheme 1—Step 5 (Example 2):

N-(5-bromo-1,3-thiazol-2-yl)-N-[4-(4-methylpiperazin-1-yl)pyridin-2-yl]acetamide from the foregoing step (0.1 g, 0.25 mmol), [3-(acetylamino)phenyl]boronic acid (112 mg, 0.63 mmol) and K₂CO₃ (174 mg, 1.3 mmol) [alternatively K₃PO₄ could be used] were dissolved in dioxane (3 ml). The mixture was degassed with a stream of N₂ for 5 minutes and then Pd(PPh₃)₄ (29 mg, 0.03 mmol) was added. The mixture was heated in the microwave to 150° C. for 2100 seconds, 0.2 ml of water was added and mixture heated to 150° C. for a further 1000 seconds. The solvent was evaporated and the residue purified by chromatography on silica gel eluting with a gradient from 5% (2.0N NH₃ in MeOH)/DCM to 5% (2.0N NH₃ in MeOH): 2% MeOH: 93% DCM to afford 14 mg of the desired product: m/z (ES⁺) 409 (MH⁺).

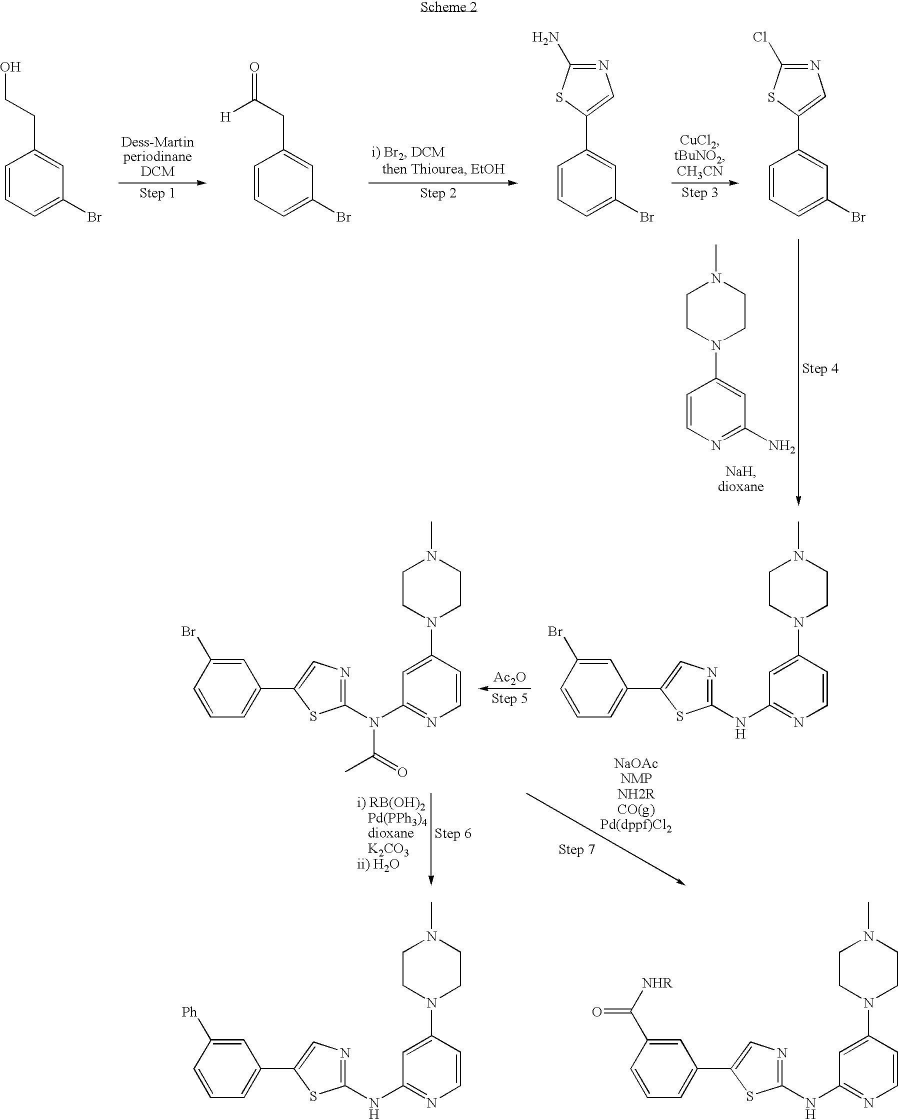

Scheme 2—Step 1:

2-(3-Bromophenyl)ethanol (3.5 ml, 25 mmol) was dissolved in DCM (50 ml) and Dess-Martin periodinane (13 g, 0.03 mol) was added. The mixture was stirred at room temperature for 4 hours. The solvent was then partially evaporated and the residue filtered through a pad of Celite©. Purification by chromatography on silica gel eluting with 10% Ethyl Acetate/Hexane afforded 5 g (98%) of the aldehyde as a colourless oil: δ_{H} (360 MHz, CDCl₃): 9.78 (1H, s), 7.48 (1H, d, J=8.0 Hz), 7.42 (1H, s), 7.23-7.26 (1H, m), 7.18 (1H, d, J=7.6 Hz), 3.71 (2H, d, J=2.0 Hz).

Scheme 2—Step 2:

A solution of bromine (1.3 ml, 25 mmol) in DCM (5 ml) was slowly added to a −10° C. cooled solution of (3-bromophenyl)acetaldehyde from the foregoing step (5 g, 25 mmol) in DCM (35 ml). The resulting solution was allowed to slowly reach room temperature and then stirred for 2 hours. It was then quenched with a saturated solution of NaHCO₃ and diluted with DCM (50 ml). The organic layer was dried over MgSO₄, concentrated and dissolved in ETOH (50 ml). After addition of thiourea (3.8 g, 50 mmol), the mixture was heated to reflux for 6 h. The solvent was evaporated under reduced pressure and the residue partitioned between EtOAc (30 ml) and sat NaHCO₃ (20 ml). The organic layer was washed with water (20 ml), dried over MgSO₄ and concentrated under reduced pressure. Crystallization from MeOH/H₂O afforded 4.6 g (73%) of the title compound as a pale orange solid: m/z (ES⁺) 255, 257 (MH⁺).

Scheme 2—Step 3:

CuCl₂ (2.9 g, 22 mmol) was suspended in dry CH₃CN (50 ml). ^{t}BuNO₂ (3.24 ml, 27 mmol) was added over 10 min followed by 5-(3-bromophenyl)-1,3-thiazol-2-amine from the foregoing step (4.6 g, 18 mmol) in CH₃CN (20 ml). It was stirred for 3 hours at room temperature with the reaction mixture turning from green to a brown colour. It was then poured into 0.5N HCl and diluted with ethyl acetate (50 ml). The organic layer was washed with water (30 ml), dried over MgSO₄ and concentrated under reduced pressure. Purification by chromatography on silica gel eluting with 20% Ethyl Acetate/Hexane afforded 3.8 g (76%) of the desired 5-(3-bromophenyl)-2-chloro-1,3-thiazole: δ_{H} (400 MHz, CDCl₃): 7.76 (1H, s), 7.52 (1H, d, J=8.0 Hz), 7.43 (1H, d, J=7.8 Hz), 7.33-7.29 (1H, m).

Scheme 2—Step 4-6:

The 5-(3-bromophenyl)-2-chloro-1,3-thiazole from the foregoing step was reacted according to scheme 1—step 2, then acetylated according to scheme 1—step 4 and finally reacted under the Suzuki conditions described in scheme 1—step 5.

Scheme 2—Step 7 (Example 17):

N-(5-bromo-1,3-thiazol-2-yl)-N-[4-(4-methylpiperazin-1-yl)pyridin-2-yl]acetamide from scheme 2—step 5 (0.15 g, 0.32 mmol), NaOAc (52 mg, 0.64 mmol) and ethane-1,2-diamine (0.2 ml, 3.2 mmol) were dissolved in NMP (2 ml). The mixture was degassed for 5 minutes with a stream of N₂ and then Pd(dppf)Cl₂ (18 mg, 0.02 mmol) was added. CO gas was bubbled through for 10 minutes until saturation of the atmosphere and then the mixture was heated to 100° C. in a sealed tube overnight. Evaporation of the solvent, purification by chromatography on silica gel eluting with a gradient from 5% (2.0N NH₃ in MeOH)/DCM to 5% (2.0N NH₃ in MeOH): 2% MeOH: 93% DCM followed by trituration from MeOH/Et₂O afforded 46 mg of the desired compound: m/z (ES⁺) 438 (MH⁺).

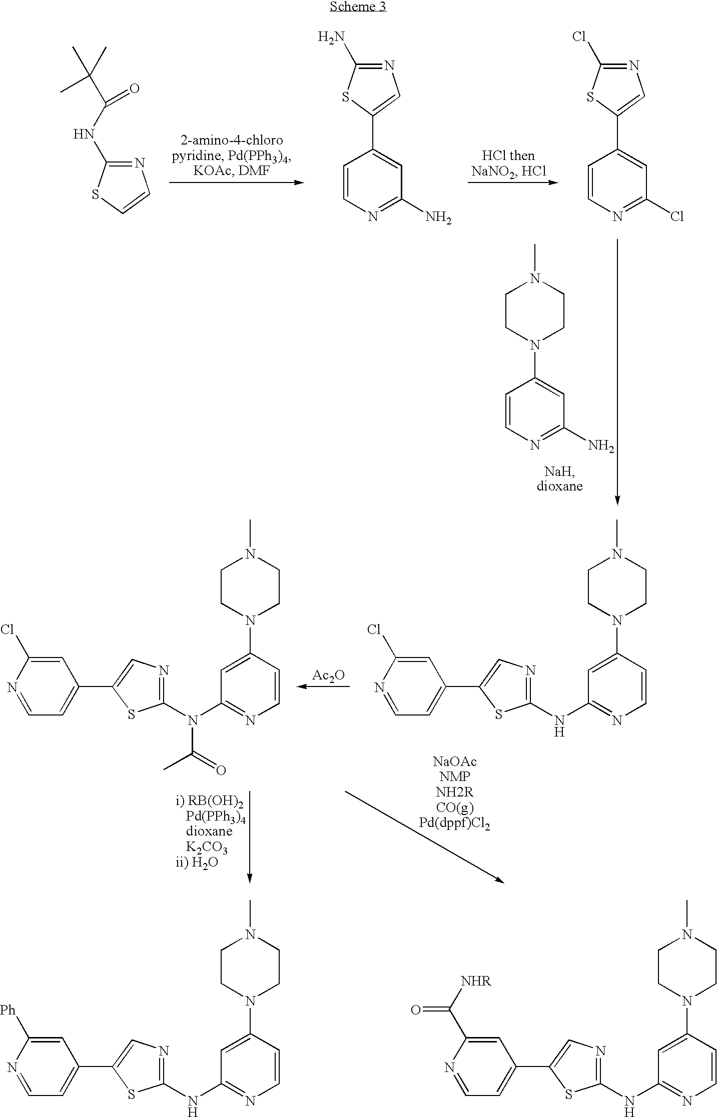

Scheme 3—Step 1

A solution of 2,2-dimethyl-N-1,3-thiazol-2-ylpropanamide (prepared as in Scheme 4—step 1, 9.6 g, 52 mmol), 2-amino-4-chloropyridine (10 g, 78 mmol), KOAc (15.3 g, 0.16 mol) in N,N-dimethylacetamide (60 ml) was degassed for 5 min with a stream of N₂. Pd(PPh₃)₄ (3 g, 2.6 mmol) was added and the mixture was heated to 125° C. in a sealed tube for 12 h. After evaporation of the solvent, the residue was dissolved in ethyl acetate (200 ml) and filtered. Purification by chromatography on silica gel eluting with ethyl acetate followed by 5% MeOH/DCM afforded 5.5 g (38%) of the desired intermediate: m/z (ES⁺) 277 (MH⁺).

Scheme 3—Step 2

N-[5-(2-aminopyridin-4-yl)-1,3-thiazol-2-yl]-2,2-dimethylpropanamide from the foregoing step (8.0 g, 29 mmol) was suspended in conc HCl (50 ml) and heated to reflux temperature for 5 h. Evaporation of the solvent and trituration of the residue with ethyl acetate gave the desired 4-(2-amino-1,3-thiazol-5-yl)pyridin-2-amine dihydrochloride which was suspended in conc HCl/water (50 ml/15 ml) and cooled to 0° C. NaNO₂ (3.0 g, 43.5 mmol) in water (15 ml) was added dropwise over 20 minutes. The mixture was stirred at 0° C. for a further 15 mins and then at room temperature for 1½ h. It was then basified to pH 8 with solid NaHCO₃ and extracted with DCM (10 ml×4). Organics were dried over MgSO₄ and concentrated under reduced pressure. Purification by chromatography on silica gel eluting with 50% ethyl acetate/hexane afforded 560 mg of the desired 2-chloro-4-(2-chloro-1,3-thiazol-5-yl)pyridine (8%): m/z (ES⁺) 231, 233 (MH⁺).

2-Chloro-4-(2-chloro-1,3-thiazol-5-yl)pyridine was elaborated to final products using procedures analogous to those shown in Scheme 2—steps 4-7.

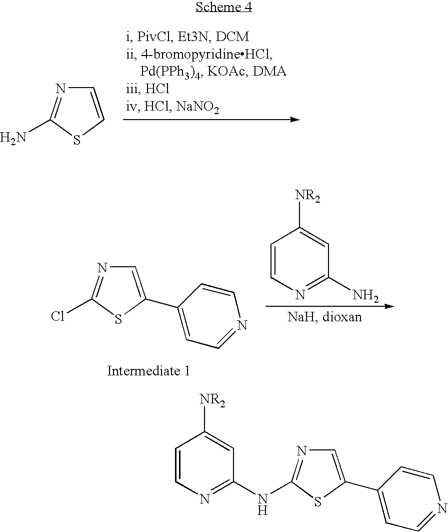

Scheme 4—Step 1

To a suspension of 2-aminothiazole (1.0 g, 10.0 mmol) in DCM (25 ml) at 0° C. was added triethylamine (1.67 ml, 11.98 mmol) then pivaloyl chloride (1.35 ml, 10.98 mmol). The cooling bath was removed, the reaction stirred for 30 minutes then the solvent removed in vacuo. The residue was taken up in EtOAc/hexanes and filtered through a plug of silica gel. Evaporation afforded the desired protected product (1.83 g) used without further purification.

Scheme 4—Step 2

To a solution of the amide from the foregoing step (0.28 g, 1.54 mmol) in dimethyl acetamide (4 ml) in a thick-walled vial was added 4-bromopyridine.HCl (0.25 g, 1.28 mmol), Pd(PPh₃)₄ (74 mg, 5 mol %) and potassium acetate (0.38 g, 3.86 mmol). The vial was sealed and heated to 150° C. for 18 hours, then cooled, diluted with EtOAc and filtered through a plug of Celite©. The filtrate was concentrated in vacuo and purified by column chromatography (silica gel; 60-75% EtOAc:hexanes eluent) to afford the desired 2,2-dimethyl-N-(5-pyridin-4-yl-1,3-thiazol-2-yl)propanamide (0.28 g).

Scheme 4—Step 3

2,2-Dimethyl-N-(5-pyridin-4-yl-1,3-thiazol-2-yl)propanamide (0.28 g, 1.07 mmol) from the foregoing step was taken up in 3M HCl (10 ml) and heated to reflux for 4 hours. On cooling, solvent was removed in vacuo and the residue taken up in EtOAc. A solid separated which was filtered and washed with further EtOAc to afford the desired aminothiazole derivative (0.20 g).

Scheme 4—Step 4

The aminothiazole derivative from the foregoing step (97 mg, 0.45 mmol) was taken up in 1 ml of an 80:20 mixture of conc. HCl:water and cooled to 0° C. To this was added a solution of sodium nitrite (1.3 ml of a 50 mg/ml solution, 0.91 mmol) and the mixture stirred at 0° C. for 2 hours then at 50° C. for 3 hours. On cooling, the mixture was neutralized by the addition of solid sodium bicarbonate and the resulting cloudy solution extracted with DCM (×3). The combined organics were dried (MgSO₄) and evaporated to afford the desired Intermediate 1 (68 mg).

Scheme 4—Step 5

Intermediate 1 was reacted with 2-amino-4-(dialkylamino)pyridine (prepared from 4-chloro-2-aminopyridine and the appropriate dialkylamine as in Scheme 1—step 1) using the procedure of Scheme 1—step 2 to afford the desired products.

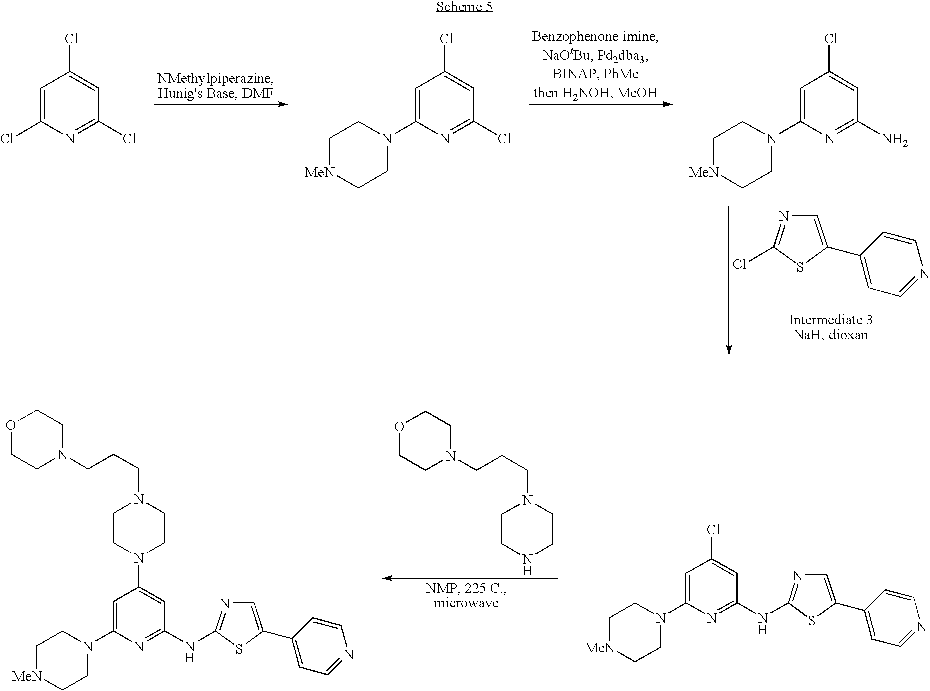

Scheme 5—Step 1

2,4,6-trichloropyridine (10 g, 47 mmol), 1-methylpiperazine (5.2 ml, 47 mmol) and Hunig's base (8.9 ml, 51 mmol) were dissolved in DMF (30 ml). The reaction mixture was stirred at room temperature for 48 h. The solvent was removed under reduced pressure and the residue dissolved in water, basified with NaHCO₃ and extracted with ethyl acetate (3×20 ml). The organics were dried over Na₂SO₄ and the removal of the solvent under reduced pressure delivered 7.15 g (62%) of the title compound as a colourless solid (containing an amount of regioisomeric impurity): m/z (ES⁻) 246, 248 (MH⁻).

### Scheme 5—Step 2

1-(4,6-dichloropyridin-2-yl)-4-methylpiperazine from the foregoing step (7.14 g, 29 mmol) was dissolved in toluene (90 ml) and benzophenone imine (5.9 ml, 35 mmol), NaO^{t}Bu (4.7 g, 49 mmol) 1.3 mmol), Pd₂(dba)₃ (1.3 g, 1.5 mmol) and BINAP (1.38 g, 2 mmol) were added. The mixture was degassed for 5 minutes with a stream of nitrogen and then heated to reflux for 3 h. After cooling to room temperature, the mixture was concentrated and the residue dissolved in MeOH (100 ml). Hydroxylamine (7 ml) was added and the mixture stirred for 12 h at room temperature. Removal of the solvent and purification by chromatography on silica gel eluting with a gradient 1-8% (2N NH₃ in MeOH)/DCM delivered 1.28 g (20%) of the desired 4-chloro-6-(4-methylpiperazin-1-yl)pyridin-2-amine: m/z (ES⁺) 227, 229 (MH⁺).

Scheme 5—Step 3

The 4-chloro-6-(4-methylpiperazin-1-yl)pyridin-2-amine from the foregoing step was reacted with Intermediate 1 (Scheme 4) using the procedure of scheme 1—step 2 to give 4-chloro-6-(4-methylpiperazin-1-yl)-N-(5-pyridin-4-yl-1,3-thiazol-2-yl)pyridin-2-amine.

Scheme 5—Step 4

The 4-chloro-6-(4-methylpiperazin-1-yl)-N-(5-pyridin-4-yl-1,3-thiazol-2-yl)pyridin-2-amine from the foregoing step was elaborated to the final product using a procedure analogous to that shown in scheme 1—step 1.

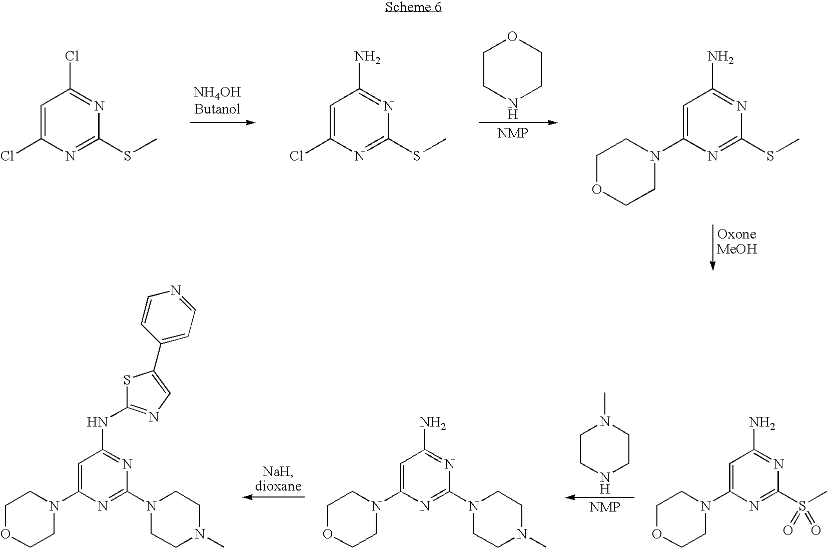

Scheme 6—Step 1

4,6-Dichloro-2-(methylthio)pyrimidine (10 g, 51 mmol) was dissolved in a mixture Butanol/NH₄OH (100 ml/50 ml). It was stirred for ½ h in a sealed tube (internal pressure 42 PSI) heated to 80° C. After cooling to RT, the organic layer was separated, dried over MgSO₄ and concentrated. 6.0 g (66%) of the desired intermediate was obtained as a colourless solid; m/z (ES⁻) 175, 177 (MH⁻).

Scheme 6—Step 2

The 6-chloro-2-(methylthio)pyrimidin-4-amine from the foregoing step was elaborated to the desired 2-(methylthio)-6-morpholin-4-ylpyrimidin-4-amine using a procedure analogous to that described in scheme 1—step 1.

Scheme 6—Step 3

2-(Methylthio)-6-morpholin-4-ylpyrimidin-4-amine from the foregoing step (0.5 g, 2.2. mmol) was dissolved in MeOH (15 ml) and oxone™ (3 g, 4.9 mmol) was added. The reaction mixture was stirred at room temperature for 12 h and then poured into a NaHCO₃ saturated solution. The compound was extracted with ethyl acetate (3×20 ml), dried over MgSO₄ and concentrated under reduced pressure. Purification by chromatography on silica gel eluting with 5% (2.0N NH₃ in MeOH)/DCM delivered 0.15 g of the desired 2-(methylsulfonyl)-6-morpholin-4-ylpyrimidin-4-amine: m/z (ES⁺) 259 (MH⁺).

Scheme 6—Step 4

The 2-(methylsulfonyl)-6-morpholin-4-ylpyrimidin-4-amine from the foregoing step was elaborated to the desired 2-(4-methylpiperazin-1-yl)-6-morpholin-4-ylpyrimidin-4-amine using a procedure analogous to that described in scheme 1—step 1.

Scheme 6—Step 5

The 2-(4-methylpiperazin-1-yl)-6-morpholin-4-ylpyrimidin-4-amine from the foregoing step was reacted with Intermediate 1 from Scheme 4 using a procedure analogous to that described in scheme 1—step 2.

The following table lists the compounds prepared by these routes. Where heteroatoms are depicted with one or more unsatisfied valencies, valency-satisfying hydrogens are implied.

| Example | Structure | ms [MH+] | Scheme |
|---|---|---|---|
| 1 | 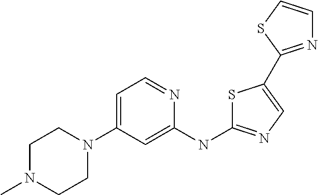 | 359 | 1 |
| 2 | 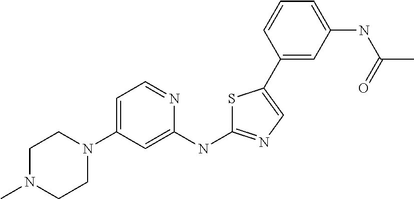 | 409 | 1 |
| 3 | 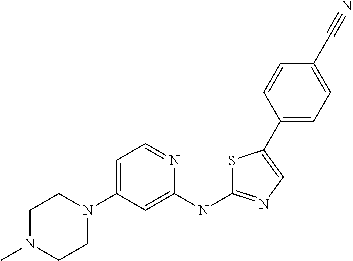 | 377 | 1 |
| 4 | 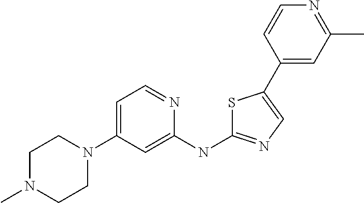 | 367 | 1 |
| 5 | 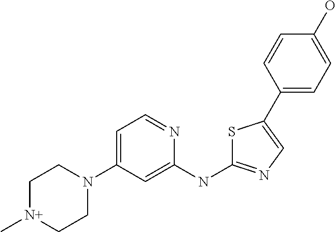 | 368 | 1 |
| 6 | 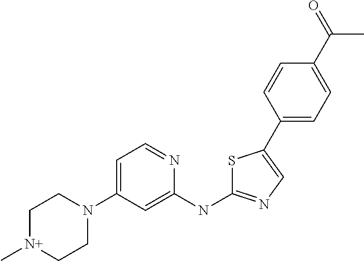 | 394 | 1 |
| 7 | 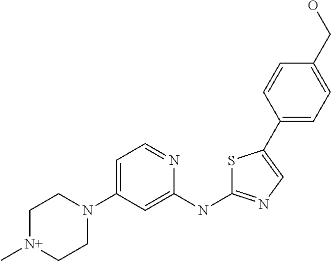 | 382 | 1 |
| 8 | 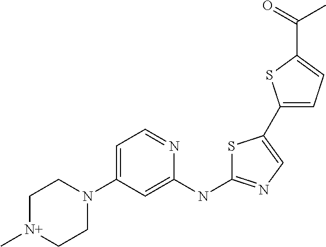 | 400 | 1 |
| 9 | 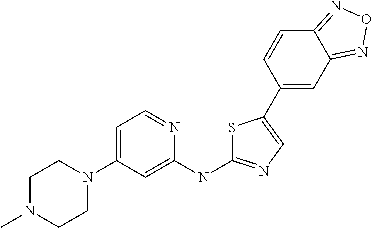 | 394 | 1 |
| 10 | 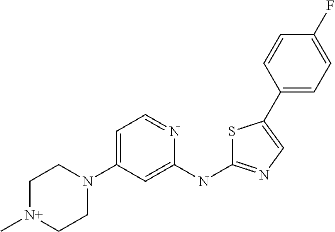 | 370 | 1 |
| 11 | 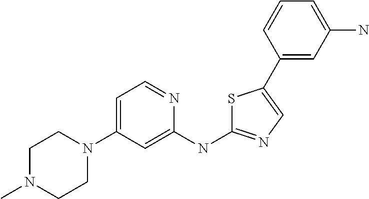 | 367 | 1 |
| 12 | 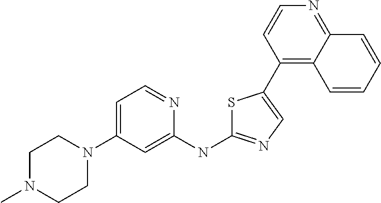 | 403 | 1 |
| 13 | 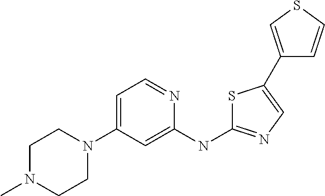 | 358 | 1 |
| 14 | 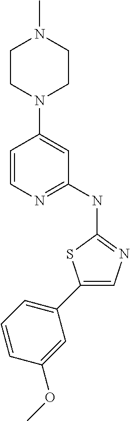 | 382 | 1 |
| 15 | 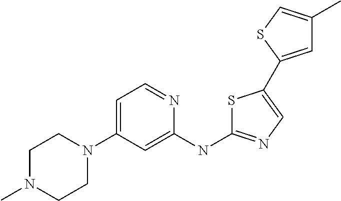 | 372 | 1 |
| 16 | 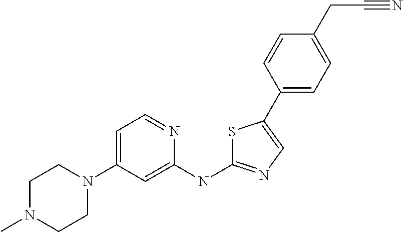 | 391 | 1 |
| 17 | 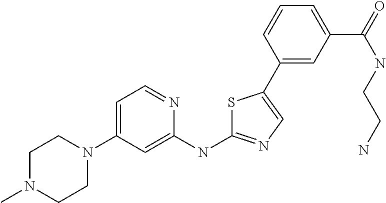 | 438 | 2 |
| 18 | 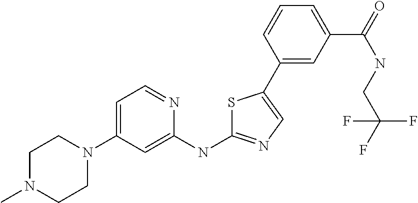 | 477 | 2 |
| 19 | 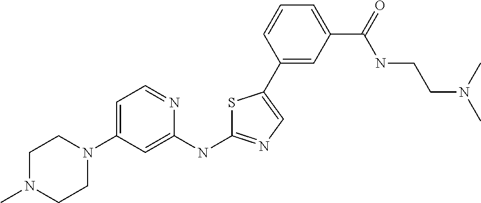 | 466 | 2 |
| 20 | 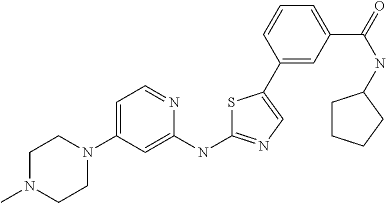 | 463 | 2 |
| 21 | 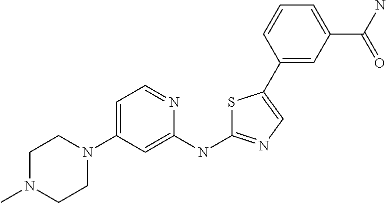 | 395 | 2 |
| 22 | 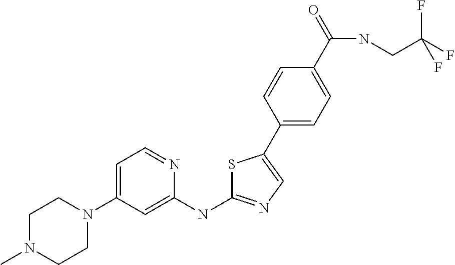 | 477 | 2 |
| 23 | 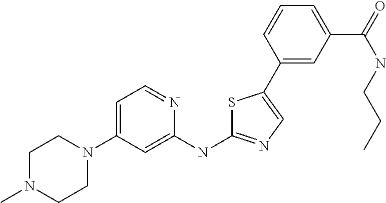 | 437 | 2 |
| 24 | 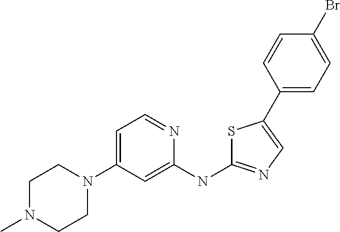 | 215 | 2 |
| 25 | 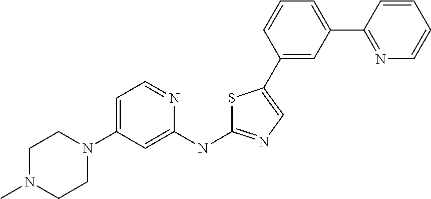 | 429 | 2 |
| 26 | 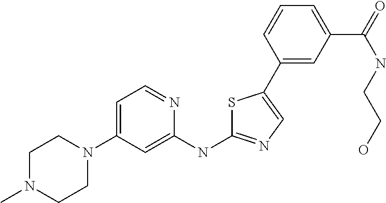 | 439 | 2 |
| 27 | 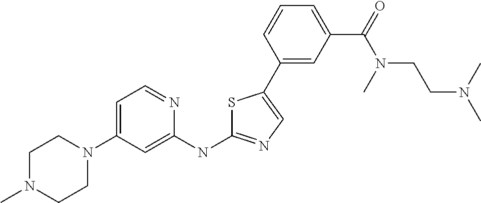 | 480 | 2 |
| 28 | 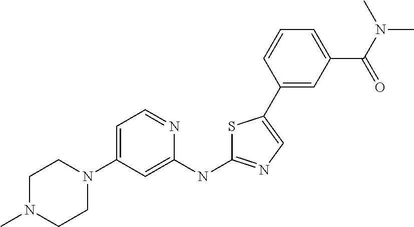 | 423 | 2 |
| 29 | 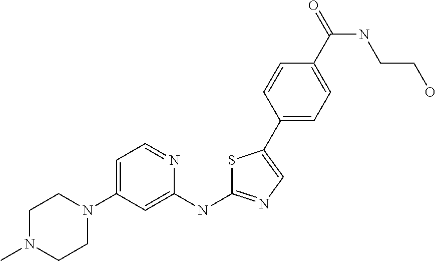 | 439 | 2 |
| 30 | 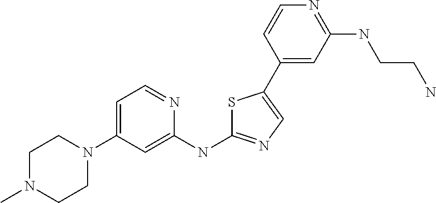 | 411 | 3 |
| 31 | 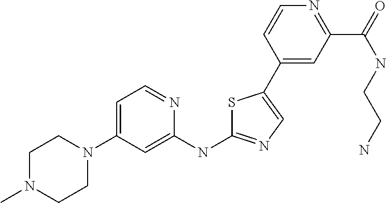 | 439 | 3 |
| 32 | 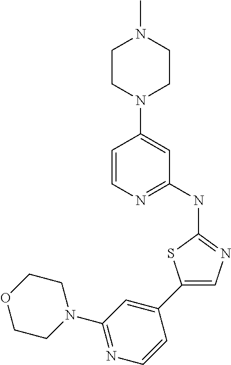 | 438 | 3 |
| 33 | 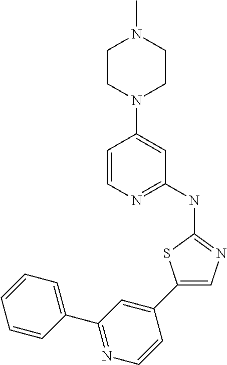 | 429 | 3 |
| 34 | 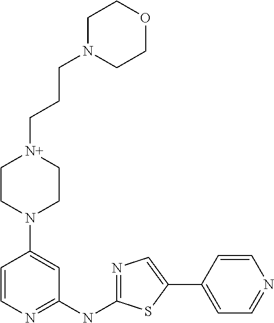 | 466 | 4 |
| 35 | 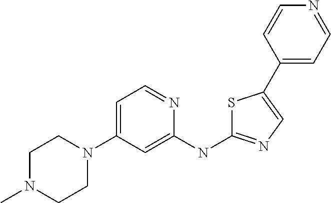 | 353 | 4 |
| 36 | 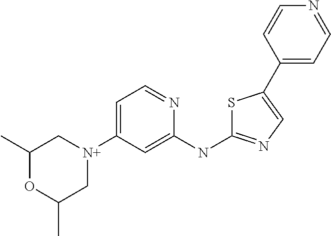 | 368 | 4 |
| 37 | 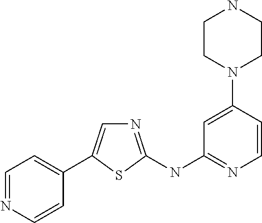 | 339 | 4 |
| 38 | 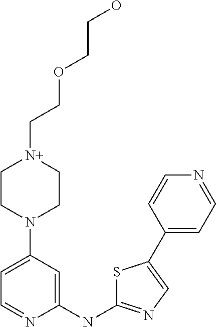 | 427 | 4 |
| 39 | 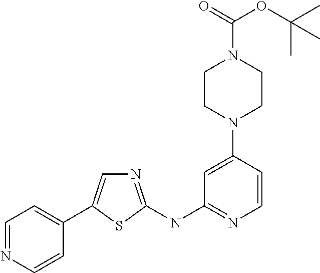 | 439 | 4 |
| 40 | 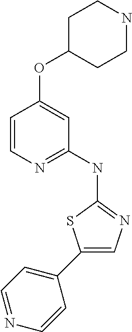 | 354 | 4 |
| 41 | 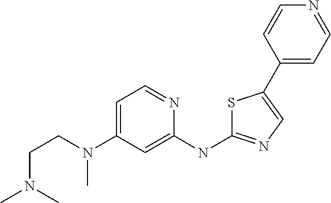 | 355 | 4 |
| 42 | 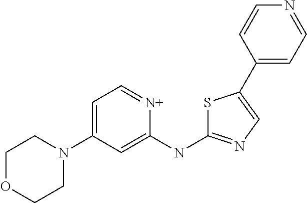 | 340 | 4 |
| 43 | 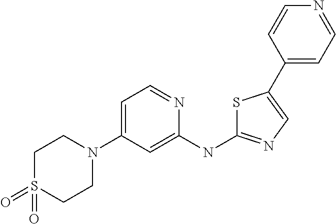 | 388 | 4 |
| 44 | 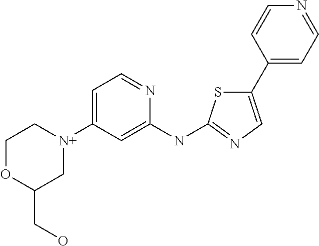 | 370 | 4 |
| 45 | 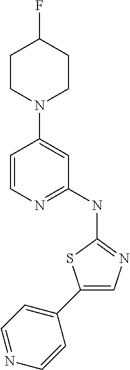 | 356 | 4 |
| 46 | 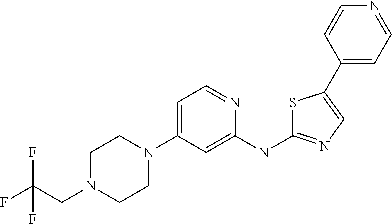 | 421 | 4 |
| 47 | 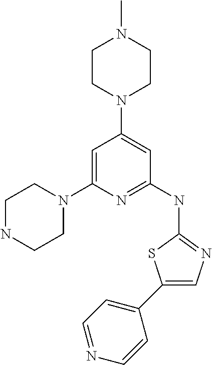 | 437 | 5 |
| 48 | 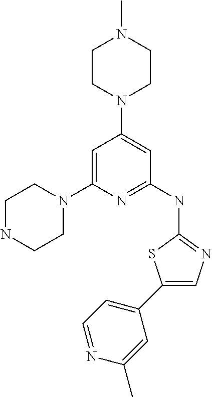 | 451 | 5 |
| 49 | 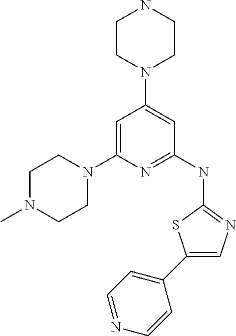 | 437 | 5 |
| 50 | 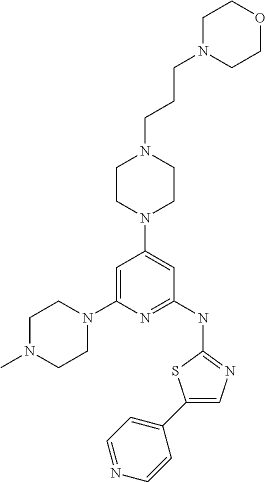 | 564 | 5 |
| 51 | 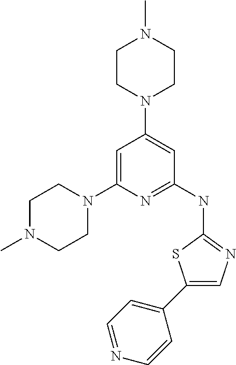 | 451 | 5 |
| 52 | 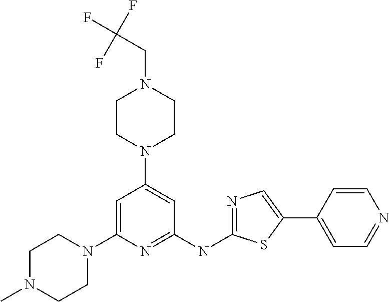 | 519 | 5 |
| 53 | 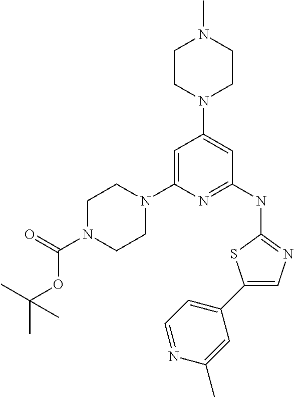 | 551 | 5 |
| 54 | 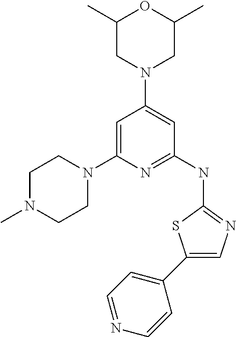 | 466 | 5 |
| 55 | 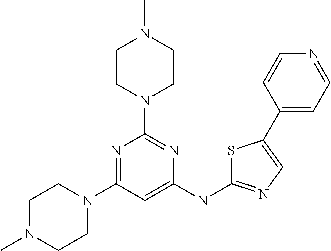 | 452 | 6 |
| 56 | 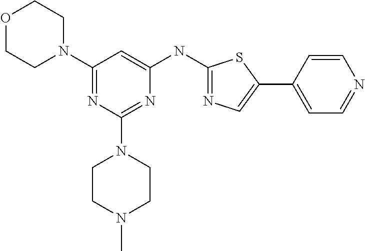 | 439 | 6 |

1. (canceled)

2. A method for treatment or prevention of a neurodegenerative disease associated with hyperphosphorylation of tau in a human patient, said method comprising administering to that patient an effective amount of a compound of formula I 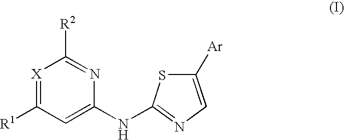
or a pharmaceutically acceptable salt or hydrate thereof; wherein:
X represents CH or N;
R¹ represents NR³R⁴ or OR⁵;
R represents H or NR³R⁴;
R³ represents H or C₁₋₄alkyl and R⁴ represents CH₂CH₂N(R⁶)₂;
or R³ and R⁴ together complete an N-heterocyclyl group which optionally bears up to 3 substituents selected from halogen, CN, CF₃, CO(O)ₙR⁶ and (CH₂)ₘ(O)ₙR⁶ where m is 1 or 2 and n is 0 or 1;
R⁵ represents C-heterocyclyl which optionally bears up to 2 substituents selected from halogen, CN, CF₃CO(O)ₙR⁶ and (CH₂)ₘ(O)ₙR⁶ where m is 1 or 2 and n is 0 or 1;
Ar represents phenyl or optionally-benzofused 5- or 6-membered heteroaryl any of which optionally bears up to 2 substituents independently represented by (CH₂)ₚY where p is 0 or 1 and Y is selected from halogen, CN, CF₃, OR⁶, COR⁶, CO₂R⁶, NR⁶COR⁶, CON(R⁶)₂, N(R⁶)₂, C₁₋₆alkyl, phenyl and pyridyl said phenyl and pyridyl optionally bearing up to 3 substituents selected from halogen, CN, OH, CF₃, C₁₋₄alkyl and C₁₋₄alkoxy;
R⁶ represents H, C₃₋₆cycloalkyl or C₁₋₆alkyl which optionally bears a substituent selected from halogen, CF₃, CN, OR⁷ or N(R⁷)₂;
or two R⁶ groups attached to the same nitrogen atom complete an N-heterocyclyl group;
R⁷ represents H or C₁₋₄alkyl, or two R⁷ groups attached to the same nitrogen atom complete an N-heterocyclyl group;
where “heterocyclyl” in each case refers to a nonaromatic ring of 5 or 6 members, of which one is N and optionally one other is N, O or S; “N-heterocyclyl” refers to a heterocyclyl group attached via a ring nitrogen atom; and “C-heterocyclyl” refers to a heterocyclyl group attached via a ring carbon atom.

3. The method according to claim 2 wherein said neurodegenerative disease associated with hyperphosphorylation of tau is selected from AD, frontotemporal dementia, Pick's disease and parkinsonism linked to chromosome 17 (FTDP-17).

4-5. (canceled)

6. A compound of formula II: 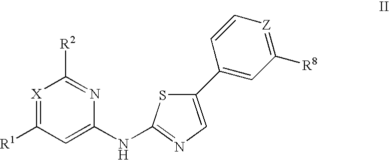
or a pharmaceutically acceptable salt or hydrate thereof; wherein;
Z represents N or CR⁹;
R⁸ represents H or (CH₂)ₚY;
R⁹ represents H or (CH₂)ₚY with the proviso that R⁸ and R⁹ do not both represent (CH₂)ₚY;
X represents CH or N;
R¹ represents NR³R⁴ or OR⁵;
R² represents H or NR³R⁴;
R³ represents H or C₁₋₄alkyl and R⁴ represents CH₂CH₂N(R⁶)₂;
or R³ and R⁴ together complete an N-heterocyclyl group which optionally bears up to 3 substituents selected from halogen, CN, CF₃, CO(O)ₙR⁶ and (CH₂)ₘ(O)ₙR⁶ where m is 1 or 2 and n is 0 or 1;
R⁵ represents C-heterocyclyl which optionally bears up to 2 substituents selected from halogen, CN, CF₃CO(O)ₙR⁶ and (CH₂)ₘ(O)ₙR⁶ where m is 1 or 2 and n is 0 or 1;
p is 0 or 1;
Y is selected from halogen, CN, CF₃, OR⁶, COR⁶, CO₂R⁶, NR⁶COR⁶, CON(R⁶)₂, N(R⁶)₂, C₁₋₆alkyl, phenyl and pyridyl, said phenyl and pyridyl optionally bearing up to 3 substituents selected from halogen, CN, OH, CF₃, C₁₋₄alkyl and C₁₋₄alkoxy;
R⁶ represents H, C₃₋₆cycloalkyl or C₁₋₆alkyl which optionally bears a substituent selected from halogen, CF₃, CN, OR⁷ or N(R⁷)₂;
or two R⁶ groups attached to the same nitrogen atom complete an N-heterocyclyl group;
R⁷ represents H or C₁₋₄alkyl, or two R⁷ groups attached to the same nitrogen atom complete an N-heterocyclyl group;
where “heterocyclyl” in each case refers to a nonaromatic ring of 5 or 6 members, of which one is N and optionally one other is N, O or S; “N-heterocyclyl” refers to a heterocyclyl group attached via a ring nitrogen atom; and “C-heterocyclyl” refers to a heterocyclyl group attached via a ring carbon atom.

7. A compound according to claim 6 wherein R¹ represents NR³R⁴ and R³ and R⁴ complete an optionally-substituted N-heterocyclyl group.

8. A compound of formula III: 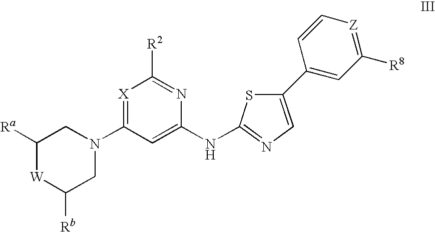
or a pharmaceutically acceptable salt or hydrate thereof; wherein:
W represents O, S, SO₂, NR^{c}, CH₂, CHF or CHCF₃;
R^{a} and R^{b} independently represent H, halogen, CN, CF₃, or (CH₂)ₘ(O)ₙR⁶;
R^{c} represents H, CF₃, CO(O)ₙR⁶ or (CH₂)ₘ(O)ₙR⁶;
X represents CH or N;
m is 1 or 2;
n is 0 or 1;
R² represents H or NR³R⁴;
R³ represents H or C₁₋₄alkyl and R⁴ represents CH₉CH₂N(R⁶)₂;
or R³ and R⁴ together complete an N-heterocyclyl group which optionally bears up to 3 substituents selected from halogen CN, CF₃, CO(O)ₙR⁶ and (CH₂)ₘ(O)ₙR⁶;
R⁶ represents H, C₃₋₆cycloalkyl or C₁₋₆alkyl which optionally bears a substituent selected from halogen, CF₃, CN, OR⁷ or N(R⁷)₂;
or two R⁶ groups attached to the same nitrogen atom complete an N-heterocyclyl group;
R⁷ represents H or C₁₋₄alkyl or two R⁷ groups attached to the same nitrogen atom complete an N-heterocyclyl group;
where “heterocyclyl” in each case refers to a nonaromatic ring of 5 or 6 members, of which one is N and optionally one other is N, O or S; “N-heterocyclyl” refers to a heterocyclyl group attached via a ring nitrogen atom; and “C-heterocyclyl” refers to a heterocyclyl group attached via a ring carbon atom
Z represents N or CR⁹;
R⁸ represents H or (CH₂)ₚY;
R⁹ represents H or (CH₂)ₚY with the proviso that R⁸ and R⁹ do not both represent (CH₂)ₚY;
p is 0 or 1;
Y is selected from halogen CN, CF₃, OR⁶, COR⁶, CO₂R⁶, NR⁶COR⁶, CON(R⁶)₂, N(R⁶)₂, C₁₋₆alkyl phenyl and pyridyl said phenyl and pyridyl optionally bearing up to 3 substituents selected from halogen CN, OH, CF₃, C₁₋₄alkyl and C₁₋₄alkoxy.

9. A compound according to claim 8 wherein R² represents NR³R⁴ and R³ and R⁴ complete an optionally-substituted N-heterocyclyl group.

10. A pharmaceutical composition comprising a compound according to claim 8 and a pharmaceutically acceptable carrier.

11-12. (canceled)

13. A pharmaceutical composition comprising a compound according to claim 6 and a pharmaceutically acceptable carrier.

14. A method for treatment or prevention of a neurodegenerative disease associated with hyperphosphorylation of tau in a human patient, said method comprising administering to that patient an effective amount of a compound of formula II as defined in claim 6, or a pharmaceutically acceptable salt or hydrate thereof.

15. The method according to claim 14 wherein said neurodegenerative disease associated with hyperphosphorylation of tau is selected from AD, frontotemporal dementia, Pick's disease and parkinsonism linked to chromosome 17 (FTDP-17).

16. A method for treatment or prevention of a neurodegenerative disease associated with hyperphosphorylation of tau in a human patient, said method comprising administering to that patient an effective amount of a compound of formula III as defined in claim 8, or a pharmaceutically acceptable salt or hydrate thereof.

17. The method according to claim 16 wherein said neurodegenerative disease associated with hyperphosphorylation of tau is selected from AD, frontotemporal dementia, Pick's disease and parkinsonism linked to chromosome 17 (FTDP-17).
